(19) United States
(12) Patent Application Publication
(10) Pub. No.: US 2026/0076667 A1
(43) Pub. Date: Mar. 19, 2026

(54) SUTURE CORRIDOR HAVING SEPARATED CONDUITS

(71) Applicant: Patrick A. MASSEY, Shreveport, LA (US)

(72) Inventor: Patrick A. MASSEY, Shreveport, LA (US)

(21) Appl. No.: 19/328,627

(22) Filed: Sep. 15, 2025

Related U.S. Application Data
(60) Provisional application No. 63/694,647, filed on Sep. 13, 2024.

(51) Int. Cl. A61B 17/04

(52) U.S. Cl. CPC A61B 17/0401; A61B 2017/0409; A61B 2017/0414; A61B 2017/0446

(57) ABSTRACT

A deformable suture corridor having separated conduits. The deformable suture corridor includes a first and a second tubular conduit. Both tubular conduits include an outer wall that is open at both ends thereof, an inner channel extending between both ends of the outer wall, and a pair of apertures in the outer wall that are in communication with the inner channel. The first conduit is coupled to the second conduit.

### CROSS-REFERENCE TO RELATED APPLICATIONS

This application claims priority to U.S. Provisional Application 63/694,647, filed Sep. 13, 2024, the entire contents of which are hereby incorporated by reference.

### BACKGROUND

Suture corridors are commonly used to secure soft tissue to bone. Deformable suture corridors have been developed to simplify insertion and provide fixation by expanding or changing shape once deployed in a pre-formed bone cavity. These anchors are typically designed to deform so as to engage the surrounding bone and to resist withdrawal of the anchor.

However, the deformation of the suture corridor can cause the suture pathway to constrict or collapse, thereby preventing the suture becoming from sliding easily through the anchor. This crimping can also damage the suture and increase the likelihood of suture breakage, complicating the surgical procedure and reducing the durability of the repair.

Accordingly, there is a need for an improved suture corridor system that avoids crimping of sutures and allows the sutures to slide easily therethrough.

### SUMMARY

According to at least one exemplary embodiment, a suture corridor having separated conduits is disclosed. The deformable suture corridor can include at least a first and a second tubular conduit. Both tubular conduits include an outer wall that is open at both ends thereof, an inner channel extending between both ends of the outer wall, and a pair of apertures in the outer wall that are in communication with the inner channel. The first conduit is coupled to the second conduit.

In an exemplary embodiment, the first conduit and the second conduit intersect each other at an intersection site. At the intersection site, the first conduit and the second conduit may be perpendicular to each other. The inner channel of the first conduit and the inner channel of the second conduit are isolated from each other at the intersection site.

In an exemplary embodiment, the first conduit and the second conduit are parallel to each other.

In an exemplary embodiment, one or both of the first conduit and the second conduit include at least one pair of apertures or at least two pairs of apertures.

According to at least one exemplary embodiment, a suture corridor system is disclosed. The suture corridor system can include an embodiment of the deformable suture corridor having separated conduits as disclosed herein, and an insertion tool. The insertion tool can include a shaft and a distal end configured to receive at least one conduit of the suture corridor therein. The suture corridor system can further include an anchor tip couplable to the distal end of the insertion tool.

In an exemplary embodiment, the distal end of the insertion tool can include a pair of parallel extensions defining a space for receiving at least one conduit of the suture corridor therebetween.

In an exemplary embodiment, the distal end of the insertion tool can include two pairs of parallel extensions, arranged in orthogonal planes and defining a space for receiving two conduits of the suture corridor therebetween.

### BRIEF DESCRIPTION OF THE FIGURES

Advantages of embodiments of the present invention will be apparent from the following detailed description of the exemplary embodiments. The following detailed description should be considered in conjunction with the accompanying figures in which:

FIGS. 1A-1B show an exemplary embodiment of a suture corridor.

FIGS. 2A-2B show another exemplary embodiment of a suture corridor.

FIGS. 3A-3D show further exemplary embodiments of a suture corridor.

FIGS. 4A-4B show another exemplary embodiment of a suture corridor.

FIGS. 5A-5B show an exemplary embodiment of an insertion tool.

FIGS. 6A-6B show another exemplary embodiment of an insertion tool.

FIGS. 7A-7B show an exemplary embodiment of an anchor tip.

FIGS. 8A-8B show another exemplary embodiment of an anchor tip.

FIGS. 9A-9D show insertion into tissue of an exemplary embodiment a suture corridor.

FIGS. 10A-10C show further exemplary embodiments of a suture corridor.

FIGS. 11A-11C show exemplary steps for suture passage through an exemplary embodiment of a suture corridor.

FIGS. 12A-12C show exemplary steps for suture passage through an exemplary embodiment of a suture corridor after the suture corridor has been inserted into a hole in bone.

### DETAILED DESCRIPTION

Aspects of the invention are disclosed in the following description and related drawings directed to specific embodiments of the invention. Those skilled in the art will recognize that alternate embodiments may be devised without departing from the spirit or the scope of the claims. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiment are not necessarily to be construed as preferred or advantageous over other embodiments. Moreover, the terms “embodiments of the invention”, “embodiments” or “invention” do not require that all embodiments of the invention include the discussed feature, advantage or mode of operation.

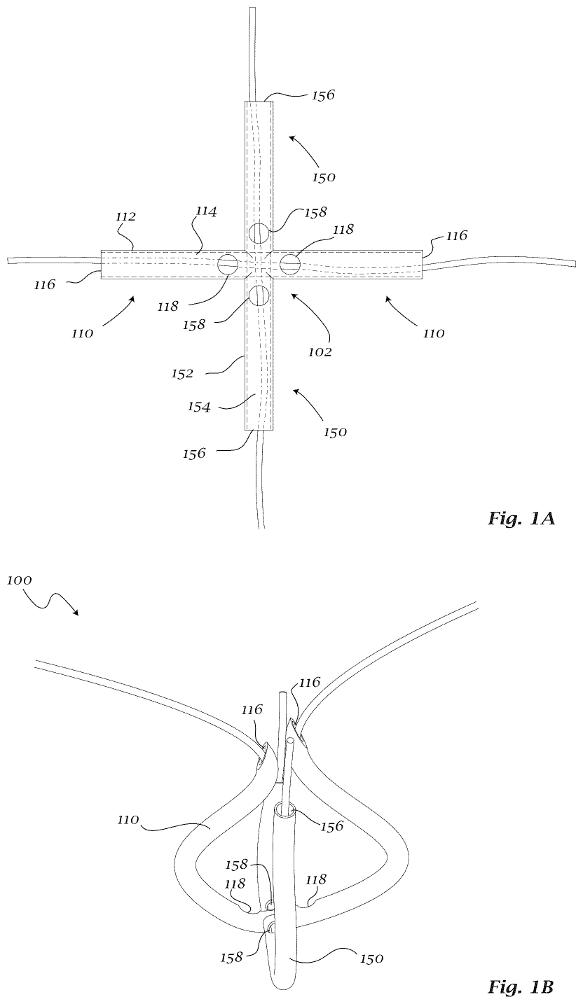

According to an exemplary embodiment as shown in FIGS. 1A-1B, a deformable suture corridor 100 having separated conduits is disclosed. Suture corridor 100 can include a first conduit 110 and a second conduit 150. First conduit 110 is formed as a tubular conduit having an outer wall 112 that is open at both ends thereof, and that encloses an inner channel 114 extending between both ends of outer wall 112. Inner channel 114 may be in communication with the external environment at both ends via terminal openings 116. Similarly, second conduit 150 is formed as a tubular conduit having an outer wall 152 that is open at both ends thereof, and that encloses an inner channel 154 extending between both ends of outer wall 152. Inner channel 154 may be in communication with the external environment at both ends via terminal openings 156.

First conduit 110 and second conduit 150 may be coupled to each other at one or more locations. In the exemplary embodiment of suture corridor 100, first conduit 110 and second conduit 150 may be coupled to each other and intersect each other at an intersection site 102. In some embodiments, at intersection site 102, first and second conduits 110, 150, may be oriented perpendicularly to each other. Further, at intersection site 102, inner channels 114, 154 of conduits 110, 150 are not in communication with each other; rather, they may be separated by a wall, membrane, or other form of barrier that prevents communication between the conduits and any contents thereof. In some embodiments, the barrier may be part of outer wall 112 or outer wall 152. In other embodiments, the barrier may be provided as an additional structure at intersection site 102. In yet other embodiments, at intersection site 102, inner channels 114, 154 of conduits 110, 150 may be in communication with each other.

First conduit 110 and second conduit 150 may be configured for various purposes. For example, first conduit 110 may be configured for knotless fixation of tissue, while second corridor 150 may allow for a sliding suture to be disposed therein, or vice versa. Additionally or alternatively, first conduit 110 may be pre-loaded with a device for passing suture, for example a wire or suture with a loop, while second conduit 150 may be preloaded with one or more sliding sutures, or vice versa. It should be appreciated that any other configuration or contents for conduits 110, 150 may be contemplated and provided as desired, and that no particular corridor is limited to any particular purpose, configuration, or contents. In any case, barrier 106 between inner channels 114, 154 can serve to prevent interaction between the contents of the inner channels, e.g., a sliding suture disposed in channel 154 would not contact, and not cause friction with, a suture disposed in channel 114.

As a non-limiting example, sutures attached to tissue may be passed through the first conduit 110, and sliding sutures may be provided in the second conduit 150. After insertion of suture corridor 100 into bone or tissue, as described further below, pulling on the tissue sutures through first conduit 110 can reduce tissue to bone. Subsequently, pulling on the sliding suture on the second conduit 150 can deform suture corridor 100, thereby creating friction to lock the sutures in first conduit 110. However, the sliding sutures in second conduit 150 can remain free to slide.

In a further exemplary embodiment of suture corridor 100, one or both of first conduit 110 and second conduit 150 may be provided with a pair of first apertures 118, 158, defined in outer walls 112, 152, respectively, and allowing communication between channels 114, 154, respectively, and the external environment. In an exemplary embodiment, first apertures 118, 158 may be provided proximate intersection site 102. In a further exemplary embodiment, when suture corridor 100 is in a non-deformed state and conduits 110, 150 are disposed in the same plane, first apertures 118, 158 may face in the same direction. In a yet another exemplary embodiment, when suture corridor 100 is in a non-deformed state and conduits 110, 150 are disposed in the same plane, first apertures 118, 158 may face in opposite directions.

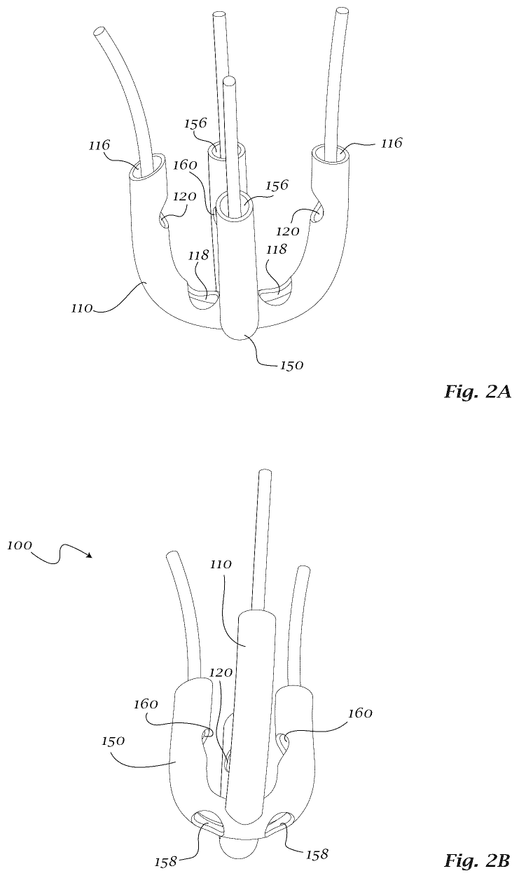

In a further exemplary embodiment of suture corridor 100, as shown in FIGS. 2A-2B, one or both of first conduit 110 and second conduit 150 may be provided with a pair of second apertures 120, 160, defined in outer wall 112, 152, respectively, and allowing communication between channels 114, 154, respectively, and the external environment. In an exemplary embodiment, second apertures 120, 160 may be provided in any desired location along conduits 110, 120, and may be disposed symmetrically with respect to intersection site 102. In further exemplary embodiments, when suture corridor 100 is in a non-deformed state and conduits 110, 150 are disposed in the same plane, one or both pairs of second apertures 120, 160 may face in the same direction as the pairs of first apertures 118, 158, or may face in the opposite direction. Other configurations and locations for the first pairs of apertures 118, 158, as well as the second pairs of apertures 120, 160, may be contemplated and provided as desired.

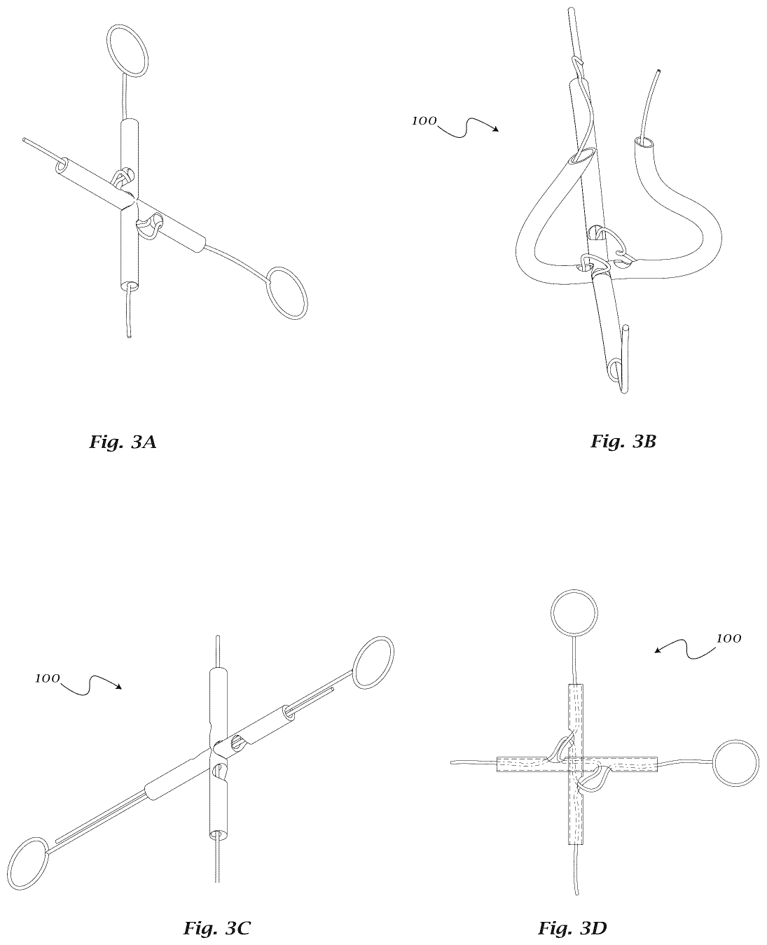

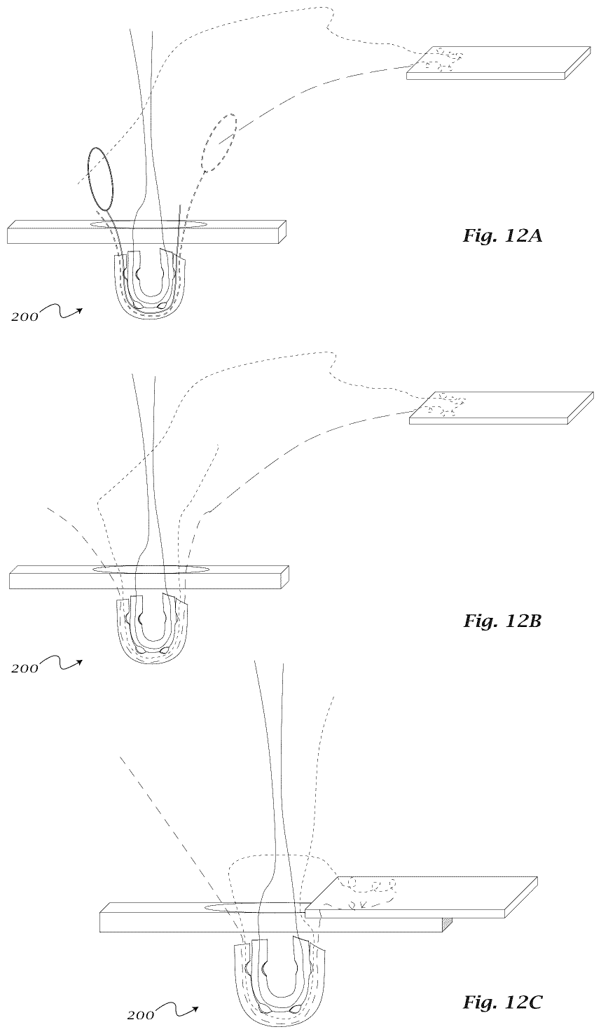

The first and second pairs of apertures can allow for diverse suture configurations. For example, sutures may be passed out of an aperture in a conduits and then into another aperture in the conduits. As another example, sutures may be passed out of an aperture in one conduits and then into an aperture in the other conduits. As further examples, a sliding suture of one conduits may be combined at the ends of the conduits to join the suture ends into a single suture, or a sliding suture of one conduits may have both ends passed into the other conduit to create a locking double loop that causes expansion of both conduits. Such a double loop may be used to facilitate reducing tendon, ligament, or tissue to bone or tissue. Exemplary suture configurations are shown in the Figures. However, other suture configurations may also be contemplated and provided as desired. FIGS. 3A-3D show non-limiting examples of embodiments of suture corridor 100 with various suture configurations. FIGS. 11A-11C, in turn, show exemplary steps for suture passage through an exemplary embodiment of a suture corridor.

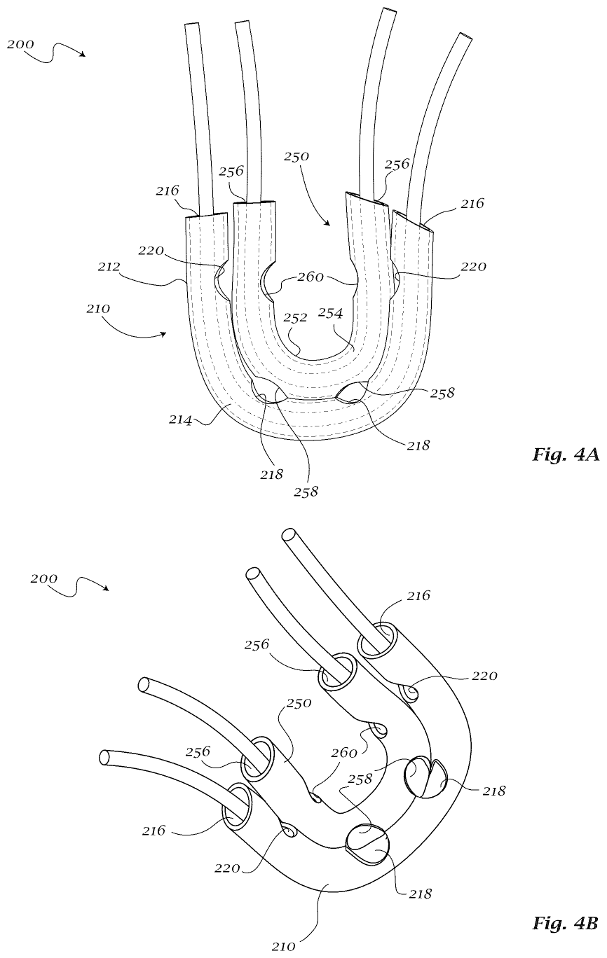

According to another exemplary embodiment, as showin in FIGS. 4A-4B, a deformable suture corridor 200 having separated conduits is disclosed. Suture corridor 200 can include a first conduit 210 and a second conduit 250. First conduit 210 is formed as a tubular conduit having an outer wall 212 that is open at both ends thereof, and that encloses an inner channel 214 extending between both ends of outer wall 212. Inner channel 214 may be in communication with the external environment at both ends via terminal openings 216. Similarly, second conduit 250 is formed as a tubular conduit having an outer wall 252 that is open at both ends thereof, and that encloses an inner channel 254 extending between both ends of outer wall 252. Inner channel 254 may be in communication with the external environment at both ends via terminal openings 256.

First conduit 210 and second conduit 250 may be coupled to each other at one or more locations. In the exemplary embodiment of suture corridor 200, first conduit 210 and second conduit 250 may be oriented parallel to each other, as parallel lines or parallel curves. Accordingly, inner channels 214, 254 of conduits 210, 250 are separated by outer wall 212 and outer wall 252 of the respective conduits. Outer walls 212, 252 can serve to prevent interaction between the contents of the inner channels, e.g., a suture disposed in channel 254 would not contact, and not cause friction with, a suture disposed in channel 214.

In a further exemplary embodiment of suture corridor 200, one or both of first conduit 210 and second conduit 250 may be provided with a pair of first apertures 218, 258 defined in outer walls 212, 252, respectively, and allowing communication between channels 214, 254, respectively, and the external environment. In an exemplary embodiment, second apertures 218, 258 may be provided in any desired location along conduits 210, 220. In a further exemplary embodiment, first apertures 218, 258 may face each other. In further exemplary embodiments, when suture corridor 200 is in a non-deformed state, first apertures 218, 258 may face in the same direction, or in opposite directions.

In a further exemplary embodiment of suture corridor 200, one or both of first conduit 210 and second conduit 250 may be provided with a pair of second apertures 220, 260, defined in outer wall 212, 252, respectively, and allowing communication between channels 214, 254, and the external environment. In an exemplary embodiment, second apertures 220, 260 may be provided in any desired location along conduits 210, 220. In a further exemplary embodiment, second apertures 220, 260 may face each other. In further exemplary embodiments, when suture corridor 200 is in a non-deformed state, second apertures 220, 260 may face in the same direction, or in opposite directions.

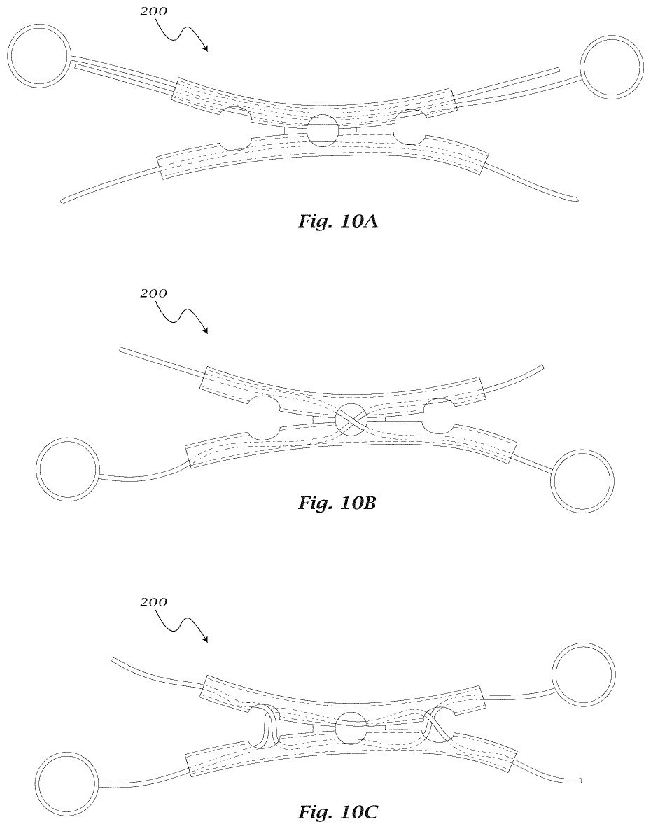

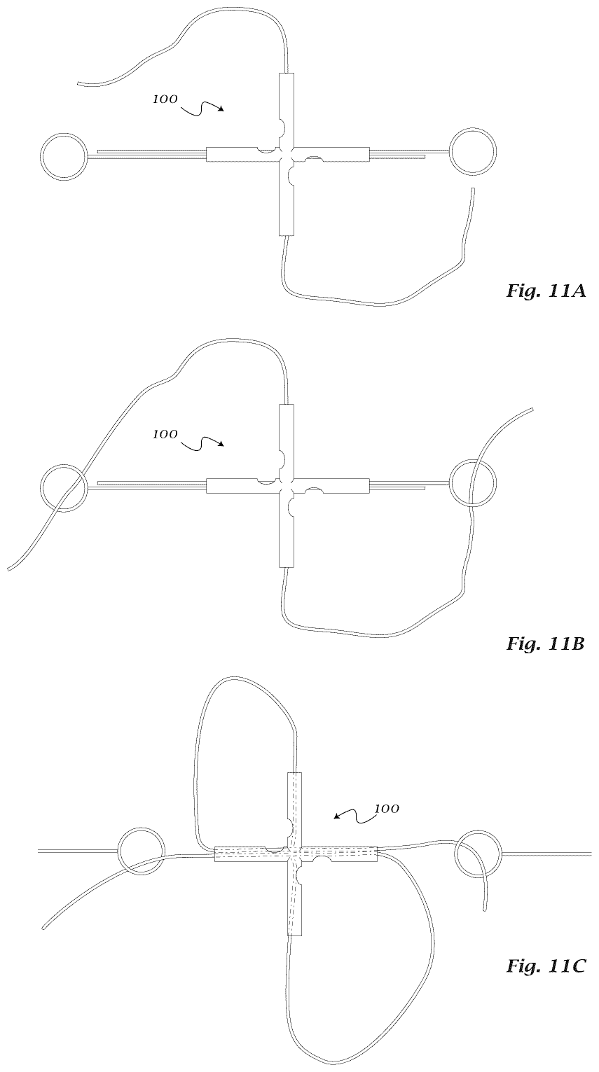

The first and second pairs of apertures can allow for diverse suture configurations. For example, sutures may be passed out of an aperture in a conduits and then into another aperture in the conduits. As another example, sutures may be passed out of an aperture in one conduits and then into an aperture in the other conduits. As further examples, a sliding suture of one conduits may be combined at the ends of the conduits to join the suture ends into a single suture, or a sliding suture of one conduits may have both ends passed into the other conduit to create a locking double loop that causes expansion of both conduits. Such a double loop may be used to facilitate reducing tendon, ligament, or tissue to bone or tissue. Exemplary suture configurations are shown in the Figures. However, other suture configurations may also be contemplated and provided as desired. FIGS. 10A-10C show non-limiting examples of embodiments of suture corridor 200 with various suture configurations.

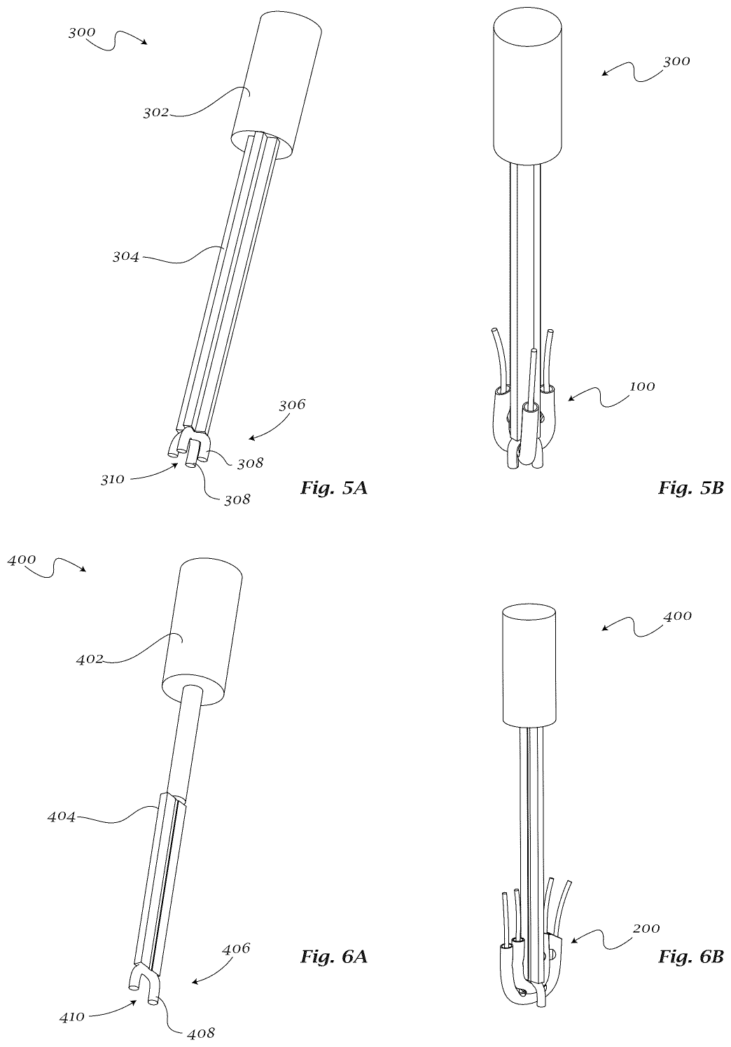

For insertion of the suture corridor into a surgical site, an insertion tool may be provided. An exemplary embodiment of insertion tool 300, as shown in FIGS. 5A-5B, may be provided for use with the embodiment of suture corridor 100. Insertion tool 300 may include a head 302, a shaft 304, and a distal end 306 adapted to receive a portion of suture corridor 100 therein. In the exemplary embodiment of insertion tool 300, distal end 306 can include two pairs of parallel extensions 308 arranged at right angles to each other, each parallel extension extending distally fromm distal end 306 and parallel to shaft 304. In some exemplary embodiments, each pair of parallel extensions 308 may be joined and provided in a U-shape. The two pairs of parallel extensions 308 can define a space 310 therebetween, into which intersection site 102 of suture corridor 100 may be inserted. Furthermore, the rounded portion of the “U” shape can provide a fit with the tubular shape of the conduits. Shaft 304 may also have a cruciform shape, with the corners of the cruciform shape providing a space in which sutures may be disposed.

For use with the embodiment of suture corridor 200, as shown in FIGS. 6A-6B, an exemplary embodiment of insertion tool 400 may be provided. Insertion tool 400 may include a head 402, a shaft 404, and a distal end 406 adapted to receive a portion of suture corridor 200 therein. In the exemplary embodiment of insertion tool 400, distal end 406 can include two parallel extensions 408 arranged opposite each other, each parallel extension extending distally fromm distal end 406 and parallel to shaft 404. In some exemplary embodiments, the pair of parallel extensions 408 may be joined and provided in a “U” shape. The pair of parallel extensions 408 can define a space 410 therebetween, into which one or both conduits 210, 250 of suture corridor 200 may be inserted. Furthermore, the rounded portion of the “U” shape can provide a fit with the tubular shape of the conduits.

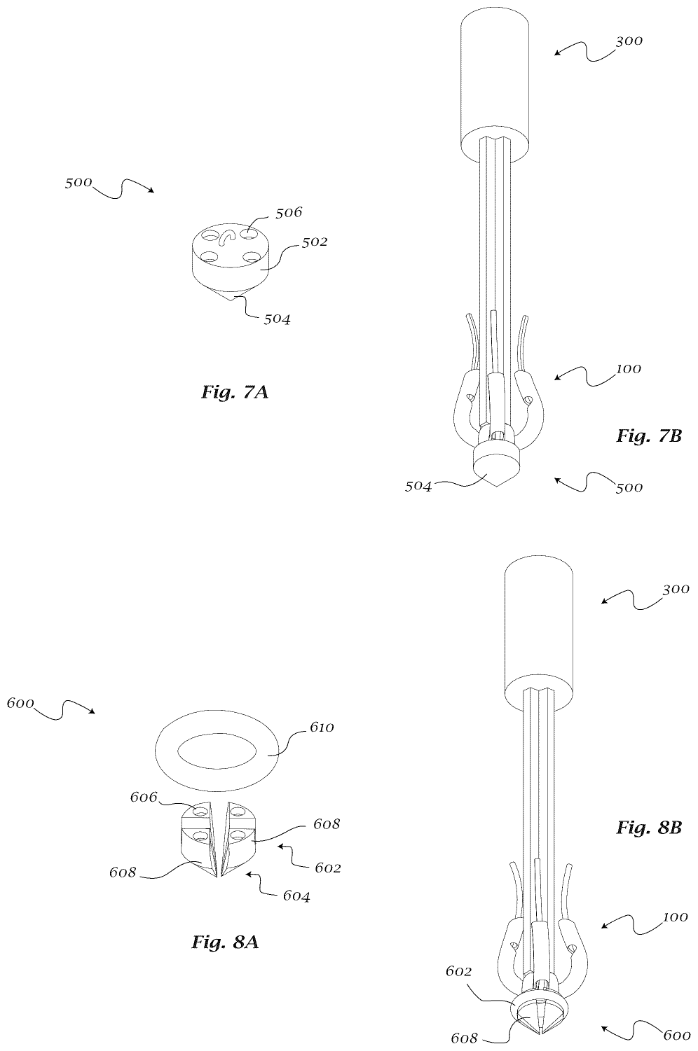

In an exemplary embodiment, as shown in FIGS. 7A-7B, a hard-tipped anchor 500 may also be provided to facilitate insertion and anchoring of suture corridor 100/200. Anchor 500 can have a cylindrical portion 502, a conical tip 504, and can be adapted to couple to the distal ends of parallel extensions 308/408 of insertion tools 300/400, for example via recesses 506.

In another exemplary embodiment, as shown in FIGS. 8A-8B, a hard-tipped anchor 600 may also be provided to facilitate insertion and anchoring of suture corridor 100/200. Anchor 600 can have a cylindrical portion 602, a conical tip 604, and can be adapted to couple to the distal ends of parallel extensions 308/408 of insertion tools 300/400, for example via recesses 606. Furthermore, the embodiment of hard-tipped anchor 600 can be subdivided into several sectors 608, for example four sectors. The multi-sector configuration of anchor 600 can facilitate ease of insertion of the anchor into bone or tissue. A ring portion 610 may optionally be provided to surround sectors 608 so as to maintain sectors 608 in proximate relation with each other while allowing for movement of sectors 608 relative to each other.

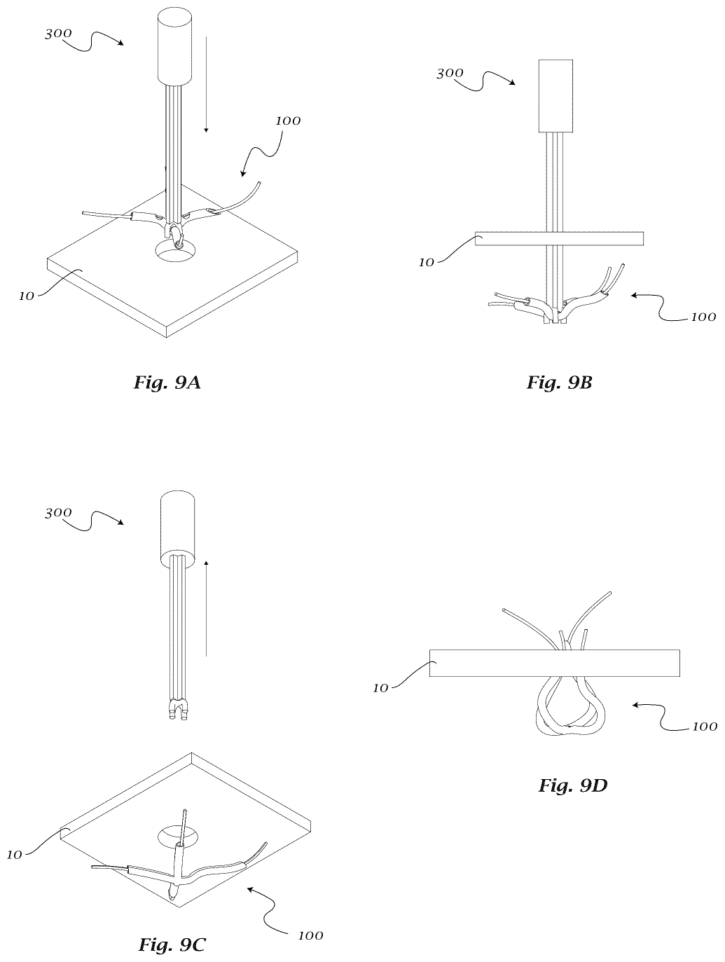

FIGS. 9A-9D show an exemplary process for insertion of an embodiment of suture corridor 100 into tissue 10 utilizing an embodiment of insertion tool 300.

FIGS. 12A-12C, in turn, show exemplary steps for suture passage through an exemplary embodiment of a suture corridor 200 after the suture corridor has been inserted into a hole in bone. FIG. 12 A shows passing of suture attached to tissue through suture loops that are inside one of the corridors. FIG. 12B shows the sutures attached to tissue, pulled through one of the corridors. FIG. 12C shows tensioning of the sutures attached to tissue with reduction of the tissue to bone and suture still free to slide in the other corridor.

It should be appreciated that the embodiments of suture corridor 100/200 disclosed herein may be inserted into bone or tissue; may be inserted through a cannulated guide; and may be loaded with many sutures in various configurations or sutures with loops to pass other sutures through the corridors. The suture may be used to affix tissue or bone to tissue or bone. Tensioning of a suture may deform portions of each corridor or may deform portions of one corridor but not deform the other corridor or portions thereof. The anchor tip may be used to insert the suture corridor system and anchor tip into bone or tissue without drilling a hole. Furthermore, embodiments of suture corridor 100/200 can be made of bioabsorbable or synthetic non-absorable material such as polyester or polyethylene. Embodiments of anchor 500 may be made of a bioabsorbable material, metal, polymer, collagen, biocomposite, or a combination thereof. Additionally all embodiments disclosed herein may be made of any material or combination of materials known in the art that enable the embodiments to function as described herein.

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed is:

1. A deformable suture corridor, comprising:
a first tubular conduit comprising an outer wall that is open at both ends thereof, an inner channel extending between both ends of the outer wall, and a pair of apertures in the outer wall that are in communication with the inner channel;
a second tubular conduit comprising an outer wall that is open at both ends thereof, an inner channel extending between both ends of the outer wall, and a pair of apertures in the outer wall that are in communication with the inner channel;
wherein the first conduit is coupled to the second conduit.

2. The suture corridor of claim 1, wherein, the first conduit and the second conduit intersect each other at an intersection site.

3. The suture corridor of claim 2, wherein, at the intersection site, the first conduit and the second conduit are perpendicular to each other.

4. The suture corridor of claim 1, wherein, the first conduit and the second conduit are parallel to each other.

5. The suture corridor of claim 2, wherein, at the intersection site, the inner channel of the first conduit and the inner channel of the second conduit are isolated from each other.

6. The suture corridor of claim 1, wherein one or both of the first conduit and the second conduit comprise two pairs of apertures.

7. The suture corridor of claim 1, wherein, in a non-deformed state of the suture corridor, the pair of apertures of the first conduit and the pair of apertures of the second conduit face in a same direction.

8. The suture corridor of claim 2, wherein the pair of apertures of the first conduit and the pair of apertures of the second conduit are located proximate the intersection site.

9. The suture corridor of claim 7, wherein, in a non-deformed state of the suture corridor, a first pair of apertures of the two pairs of apertures and a second pair of apertures of the two pairs of apertures face in a same direction.

10. The suture corridor of claim 7, wherein, in a non-deformed state of the suture corridor, a first pair of apertures of the two pairs of apertures and a second pair of apertures of the two pairs of apertures face in opposite directions.

11. A suture corridor system, comprising:
the deformable suture corridor of claim 1; and
an insertion tool comprising a shaft and a distal end configured to receive at least one conduit of the suture corridor therein.

12. The suture corridor system of claim 11, further comprising an anchor tip couplable to the distal end of the insertion tool.

13. The suture corridor system of claim 11, wherein the distal end of the insertion tool comprises a pair of parallel extensions defining a space for receiving at least one conduit of the suture corridor therebetween.

14. The suture corridor system of claim 11, wherein the distal end of the insertion tool comprises two pairs of parallel extensions, arranged in orthogonal planes and defining a space for receiving two conduits of the suture corridor therebetween.

15. A deformable suture corridor, comprising:
a first tubular conduit comprising an outer wall that is open at both ends thereof, an inner channel extending between both ends of the outer wall, and a first pair of apertures in the outer wall that are in communication with the inner channel;
a second tubular conduit comprising an outer wall that is open at both ends thereof, an inner channel extending between both ends of the outer wall, and a first pair of apertures in the outer wall that are in communication with the inner channel;
wherein the first conduit and the second conduit intersect each other at an intersection site.

16. The suture corridor of claim 15, wherein, in a non-deformed state of the suture corridor, the first pair of apertures of the first conduit and the first pair of apertures of the second conduit face in a same direction.

17. The suture corridor of claim 16, wherein:
a conduit or both conduits of the first conduit and the second conduit comprises a second pair of apertures; and
when the suture corridor is in a non-deformed state, the second pair of apertures of the conduit face in a same direction as the first pair of apertures of the conduit or in a direction opposite the first pair of apertures of the conduit.

18. A deformable suture corridor, comprising:
a first tubular conduit comprising an outer wall that is open at both ends thereof, an inner channel extending between both ends of the outer wall, and a first pair
of apertures in the outer wall that are in communication with the inner channel;
a second tubular conduit comprising an outer wall that is open at both ends thereof, an inner channel extending between both ends of the outer wall, and a first pair of apertures in the outer wall that are in communication with the inner channel;
wherein the first conduit and the second conduit are coupled to and parallel to each other.

19. The suture corridor of claim 17, wherein the first pair of apertures of the first conduit faces the first pair of apertures of the second conduit.

20. The suture corridor of claim 19, wherein:
the first conduit comprises a second pair of apertures;
the second conduit comprises a second pair of apertures; and
the second pair of apertures of the first conduit and the second pair of apertures of the second conduit face in a same direction.
